(19) United States
(12) Patent Application Publication
(10) Pub. No.: US 2013/0210759 A1
(43) Pub. Date: Aug. 15, 2013

(54) Composition for Use in the Treatment of Acne

(71) Applicant: Kenneth James, West Berkshire (GB)

(72) Inventor: Kenneth James, West Berkshire (GB)

(73) Assignee: Bio Skincare Limited, London (GB)

(21) Appl. No.: 13/877,532

(22) PCT Filed: Oct. 5, 2011
(86) PCT No.: PCT/GB2011/051894

§ 371 Date: Apr. 3, 2013

(30) Foreign Application Priority Data
Oct. 6, 2010 (GB) 1016836.7
Jun. 23, 2011 (GB) 1110659.8

(51) Int. Cl. A61K 31/7024

(52) U.S. Cl. CPC A61K 31/7024
USPC 514/53

(57) ABSTRACT

A composition comprising from 10 to 15% by weight of sucrose palmitate which may be used in the treatment of acne.

The present invention relates to the use of a sucrose fatty acid ester in the treatment of acne.

Skin conditions such as acne afflict a significant proportion of the population. A study in Germany reported acne as being prevalent in just over 25% of the population (Br J Dermatol 2001 Jul; 145(1):100-4). Acne comes in many varieties including blackheads, whiteheads and more severe forms including severe nodular acne and cystic acne. Accordingly, there is significant interest in identifying new alternative treatments for acne.

The use of fatty acids, fatty acid salts and sucrose esters in cosmetic compositions and compositions that come into contact with the skin is known.

U.S. Pat. No. 2, 733,252 discloses a process for the preparation of the fatty acid esters of lactylic acid and salts thereof in a commercial environment.

U.S. Pat. Nos. 3,098,795 and 4,422,952 disclose the use of fatty acid esters as emulsifiers in a composition.

U.S. Pat. Nos. 3,896,238, 4,150,114 and 4,046,886 disclose the use of a sucrose ester in combination with an alkyl sulfoxide or phosphine oxide in compositions for enhancing the penetration of pharmacologically active agents into the skin. Preferred sucrose esters include mono- and diacyl esters wherein the acyl substituents contain 8 to 20 carbon atoms with sucrose monooleate the most preferred.

U.S. Pat. No. 4,822,601 discloses a cosmetic base composition exhibiting therapeutic properties which comprises an acyl fatty acid lactylate ester or alkali metal salt thereof, a sucrose fatty acid ester and a solvent.

U.S. Pat. No. 4,990,501 discloses compositions for application to human and animal tissue surfaces e.g. skin. The compositions comprise an aqueous medium and a film forming component such as a sucrose ester that is at least partially soluble in water.

Surprisingly it has been found that compositions comprising a non-water-soluble sucrose fatty acid ester as the therapeutic ingredient are effective for use in the treatment of acne. In particular, these compositions may consist essentially of the non-water-soluble sucrose fatty acid ester as the only therapeutic ingredient. The composition may include further ingredients to improve its properties and ease of application.

Accordingly, the present invention provides a composition comprising from 1 to 50% by weight of a non-water-soluble sucrose fatty acid ester. Preferably the composition comprises from 2 to 40%, more preferably from 5 to 30%, more preferably from 7.5 to 25%, most preferably from 7.5 to 12.5% of the non-water soluble sucrose fatty acid ester.

In this document, the term ‘non-water-soluble’, in relation to a sucrose ester, means that a mixture of 90% water and 10% of the sucrose ester, by weight, at 20° C. does not form a single phase.

The non-water-soluble sucrose fatty acid ester is typically an ester of a saturated fatty acid having from 14 to 20 carbon atoms. Suitable fatty acid esters include sucrose myristate, sucrose palmitate and sucrose stearate. The fatty acid ester may comprise a mono acid ester, a di-acid ester, a tri-acid ester, a poly-acid ester or a mixture thereof. Sucrose palmitate is a preferred non-water-soluble sucrose fatty acid ester for use in the present invention. Sucrose palmitate is preferably either sucrose monopalmitate, sucrose dipalmitate or a mixture thereof.

In a preferred embodiment of the present invention the composition comprises from 1 to 50% by weight of a non-water-soluble sucrose fatty acid ester wherein the sucrose ester comprises at least about 70% by weight of a sucrose ester of one organic acid. For example the sucrose ester is nominally sucrose palmitate, and is made with an organic acid that is at least about 70% palmitic acid. Alternatively the sucrose ester is nominally sucrose stearate, made with an organic acid that is at least about 70% stearic acid; or the sucrose ester is nominally sucrose myristate made with an organic acid that is at least about 70% myristic acid.

In a preferred embodiment of the present invention the composition comprises from 7.5 to 25%, by weight, more preferably from 7.5 to 12% of sucrose palmitate. Preferably the therapeutic ingredient of the composition consists essentially of sucrose palmitate.

In a preferred embodiment, the present invention provides a semi-solid composition comprising emulsifier, oil and/or wax and from 10 to 15% by weight of sucrose palmitate, where the composition comprises a greater proportion of sucrose palmitate than of oil and/or wax and wherein the total amount of emulsifier in the composition is up to 20% of the composition.

The composition forms an emulsion of oily components in water. Therefore preferred emulsifiers are those which have a hydrophilic lipophilic balance (HLB) of from 12 to 18, preferably from 14 to 18. Typical emulsifiers include sorbitan fatty acid esters, ethoxylated derivatives of sorbitan fatty acid esters, alkyl polyglucosides and lactitol fatty acid ester, preferably sorbitan fatty acid esters and ethoxylated derivatives of sorbitan fatty acid esters. In addition, sucrose fatty acid esters typically have an HLB in the range 12 to 18 and are considered to be emulsifiers of the present invention.

The composition of the present invention is a stable emulsion of oil in water. The composition is a semi-solid composition such as a cream and it is not in the form of a liquid.

In a preferred embodiment of the present invention, the composition comprises up to 15% emulsifier.

The oils and waxes of the present invention include oils and waxes such as refined coconut oil, fractionated coconut oil, light mineral oil, stringy petrolatum, beeswax, beeswax substitute, lanolin, tea tree oil, lavender oil, eucalyptus oil, rosemary oil and apricot kernel oil. In a preferred embodiment the composition of the present invention comprises a ratio of sucrose palmitate to oil and/or wax of 3:1, more preferably 3:2, more preferably 2:1.

In a particularly preferred embodiment of the present invention the composition comprises up to 20% of an emulsifier with an HLB of 12-18.

In a preferred embodiment of the present invention the composition comprises a non-water-soluble sucrose fatty acid ester in combination with cosmetically and/or pharmaceutically acceptable excipients and diluents.

Surprisingly, it has been found that neither a water solubilising ester nor a film forming agent is required in the composition of the present invention. Typical further components of the composition of the present invention include oils and waxes, components to improve the skin feel of the resulting composition, and preservative. Preferably the composition comprises more non-water-soluble sucrose fatty acid ester than oil and wax for example, twice as much non-water-soluble sucrose fatty acid ester as oils and waxes.

Surprisingly, it has been found that the compositions of the present invention are effective in the treatment of acne. When applied to patients, an improvement in their skin condition was visible within as little as 1 to 2 days. Accordingly, the present invention provides a composition of the present invention for use in the treatment of acne. In particular, the present invention provides a composition of the present invention where the active ingredient consists essentially of one or more non-water-soluble fatty acid esters for use in the treatment of acne. In a preferred embodiment the present invention provides a composition where the non-water-soluble sucrose fatty acid ester consists essentially of sucrose palmitate for use in the treatment of acne. In a preferred embodiment, the present invention provides a semi-solid composition comprising emulsifier, oil and/or wax and from 10 to 15% by weight of sucrose palmitate, where the composition comprises a greater proportion of sucrose palmitate than of oil and/or wax and wherein the total amount of emulsifier in the composition is up to 20% of the composition for use in the treatment of acne.

The present invention also provides a method of treating acne, the method comprising administering to a subject in need of such treatment an effective amount of a non-water-soluble sucrose fatty acid ester, for example in a composition of the present invention. The present invention also provides the use of a non-water-soluble sucrose fatty acid ester in the preparation of a medicament for the treatment of acne.

Acne is often caused by the over-secretion of sebum. It is postulated that the treatment of the present invention is effective due to emulsifying the sebum. The emulsified sebum is then easily removed and acne then does not form.

The composition described above can be used as a preventative method of treatment for acne in that the composition is applied to the entire area that may show acne. For example, the composition may be applied once or twice a day to the entire area, e.g. the face or back of a patient.

Furthermore, the composition described above may also be used as a base layer on a face, for example, prior to the application of cosmetics, e.g foundation onto the face.

The present invention also provides a further composition for the topical treatment of acne spots. The composition comprises a non-water-soluble sucrose fatty acid ester and a water-soluble sucrose fatty acid ester. In a particularly preferred embodiment the composition comprises sucrose palmitate and sucrose oleate. This composition may be used as part of a treatment regime for acne in combination with the first composition of the invention described above.

Accordingly the present invention provides a kit comprising a) a composition comprising from 1 to 50% by weight of a non-water-soluble sucrose fatty acid ester, together with instructions for simultaneous, concurrent, separate or sequential use in combination with b) a composition comprising a non-water-soluble sucrose fatty acid ester and a water-soluble sucrose fatty acid ester for use in the treatment of acne. In a preferred embodiment of the present invention the first composition is applied to the entire area that is affected by acne and the second composition is applied to individual spots. In a preferred embodiment, the first composition is applied twice a day to the area affected by acne and the second composition is applied once a day to individual spots. In a preferred embodiment of the invention, the kit comprises a) a composition comprising from 1 to 50% by weight of sucrose palmitate, together with instructions for simultaneous, concurrent, separate or sequential use in combination with b) a composition comprising sucrose palmitate and sucrose oleate for use in the treatment of acne. In a particularly preferred embodiment, the kit comprises a) a semi-solid composition comprising emulsifier, oil and/or wax and from 10 to 15% by weight of sucrose palmitate, where the composition comprises a greater proportion of sucrose palmitate than of oil and/or wax and wherein the total amount of emulsifier in the composition is up to 20% of the composition, together with instructions for simultaneous, concurrent, separate or sequential use in combination with b) a composition comprising a non-water-soluble sucrose fatty acid ester and a water-soluble sucrose fatty acid ester for use in the treatment of acne. In a particularly preferred embodiment the kit comprises a) a semi-solid composition comprising emulsifier, oil and/or wax and from 10 to 15% by weight of sucrose palmitate, where the composition comprises a greater proportion of sucrose palmitate than of oil and/or wax and wherein the total amount of emulsifier in the composition is up to 20% of the composition, together with instructions for simultaneous, concurrent, separate or sequential use in combination with b) a composition comprising sucrose palmitate and sucrose oleate for use in the treatment of acne.

The following illustrative examples relating to formulations are made in accordance with the present invention are intended to illustrate a typical compositions and are not intended to be limiting on the scope of the invention. All materials used in the formulations are commercially available.

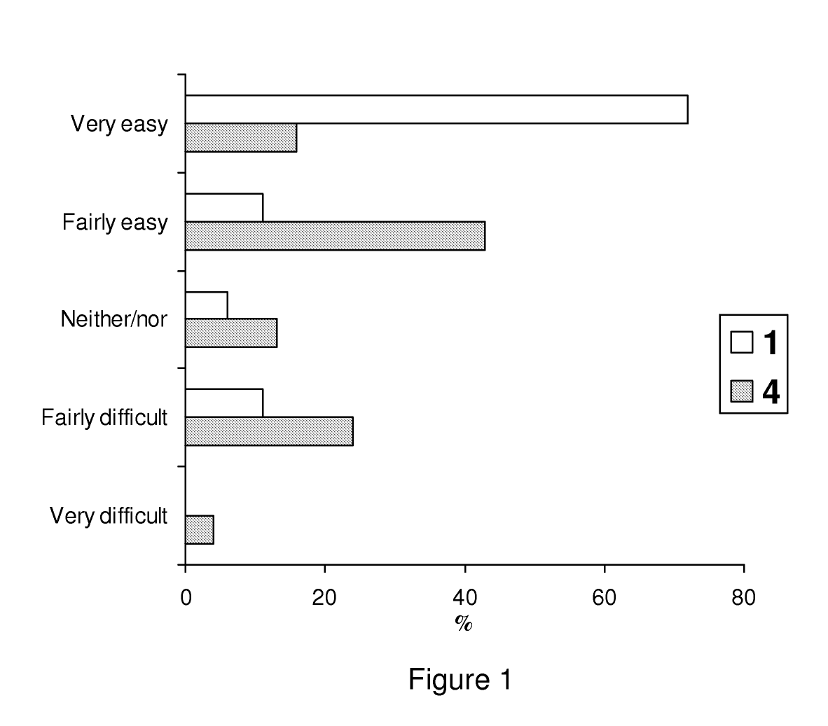

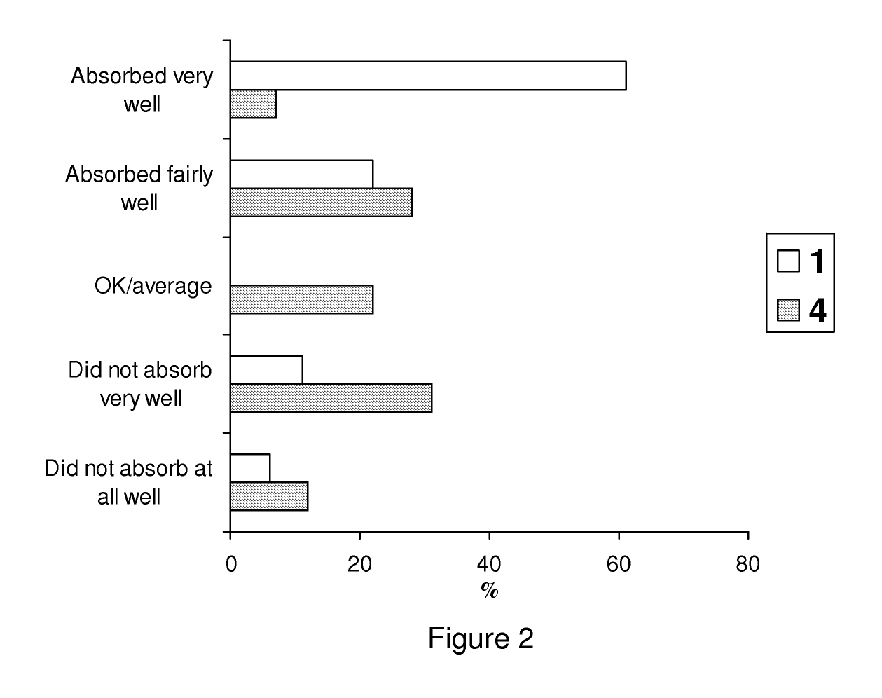

FIGS. 1 and 2 display the data obtained in example 8 in graph form.

FIG. 1 compares the ease of applying the creams of formulations 1 and 4.

FIG. 2 compares the absorption of the creams of formulations 1 and 4.

### EXAMPLE 1

Formulation 1

| Ingredient | Percent (by weight) |
|---|---|
| Sucrose mono/dipalmitate | 12 |
| Oils & waxes | 4 |
| (of which beeswax 2%, apricot kernel oil 1% and | |
| fractionated coconut oil 1%) | |
| Isopropyl myristate | 3 |
| Cetyl alcohol | 2 |
| Calcium stearate | 1 |
| Preservative | 0.3 |
| Soft water | 77.7 |

### EXAMPLE 2

Formulation 2

| Ingredient | Percent (by weight) |
|---|---|
| Sucrose mono/dipalmitate | 10 |
| Oils & waxes | 4 |
| (as beeswax 2%, apricot kernel oil 1% and | |
| fractionated coconut oil 1%) | |
| Calcium stearate | 2 |
| Isopropyl myristate | 3 |
| Cetyl alcohol | 3 |
| Preservative | 0.3 |
| Soft water | 77.7 |

Formulation 3

| Ingredient | Percent (by weight) |
|---|---|
| Sucrose palmitate | 7.2 |
| Sucrose oleate, | 5.2 |
| Sodium carboxy methyl cellulose | 5.6 |
| mono diglycerides | 2 |
| preservative | 0.3 |
| water | 79.7 |

In each of these formulations the pH of the resulting formulation was typically between 4.5-8.0. This pH range is preferred as it encompasses the skins natural pH level, and is therefore less likely to cause skin irritation.

The following illustrative examples relating to efficacy are made in accordance with the present invention and are not intended to be limiting on the scope of the invention.

### EXAMPLE 3

The composition of formulation 2 was applied to the subject's back and rubbed well into the skin. This was done before retiring for the night. This treatment process was repeated every evening for 3 days.

The subject's back was extremely spotty before treatment began. After three days treatment the spots had disappeared.

### EXAMPLE 4

The product of Formulation 2 was used for an in-home placement study conducted on a sample of adults suffering from mild acne. Respondents were given a sample tub of the cream of formulation 2 and instructed to apply the cream to the affected area twice a day. A diary sheet was placed with the cream for respondents to record their experience of the product during the trial period. Following a placement period of approximately 2 weeks a follow-up interview was conducted to assess opinions of the product.

73% of respondents noted that the product was effective.

### COMPARATIVE EXAMPLE 5

The product of Formulation 3 was used for an in-home placement study conducted on a sample of adults suffering from mild acne. Respondents were given a sample tube of the cream of formulation 3 and usage instructions and asked to apply it to the spots before retiring to bed. A diary sheet was placed with the cream for respondents to record their experience of the product during the trial period. Following a placement period of approximately 2 weeks a follow-up interview was conducted to assess opinions of the product.

53% of respondents noted that the product was effective.

### EXAMPLE 6

The products of Formulations 2 and 3 were used together in an in-home placement study conducted on a sample of adults suffering from mild acne. Respondents were given a sample tub of the cream of formulation 2 and instructed to apply the cream to the affected area twice a day. The respondents were also given a sample tub of the cream of formulation 3 and instructed to apply the cream directly to the spots before retiring to bed. A diary sheet was placed with the cream for respondents to record their experience of the product during the trial period. Following a placement period of approximately 2 weeks a follow-up interview was conducted to assess opinions of the product. 80% noted an improvement in their skin condition.

### EXAMPLE 7

The composition of Formulation 1 was used by a teenaged girl with acne that was not alleviated by treatment with antibiotics. After application of the composition of Formulation 1 once or twice daily the acne was promptly reduced and the remaining blemishes also healed. After about two months of use it was noticeable that no new spots had occurred for a significant length of time.

### EXAMPLE 8

A comparative study was carried out to compare the composition of formulation 1 with the composition of formulation 4.

Formulation 4

| Ingredient | Percent (by weight) |
|---|---|
| Sucrose mono/dipalmitate | 15 |
| Mono/diglycerides | 9 |
| Cetyl alcohol | 2 |
| Beeswax | 2 |
| Glycerol | 0.2 |
| Calcium stearate | 4 |
| Titanium dioxide | 0.08 |
| Preservative | 0.3 |
| Water | 67.67 |

Similar to formulations 1-3, this formulation also has a pH in the range 4.5-8.0.
An in-home placement study was carried out for each of these formulations. 100 respondents were given a tub of Formulation 4 for about 2 weeks and a diary sheet to record their usage and perceptions of the cream. Subsequently the respondents were given a tub of Formulation 1 for about 2 weeks and again given a diary sheet. All of the respondents suffered from some form of eczema. As well as comparing the efficacy of the formulations in the treatment of eczema, the study also resulted in data about the relative absorption, application and perception of the two formulations.
The respondents found it significantly easier to apply the composition of formulation 1 than formulation 4 and the composition of formulation 1 was perceived by the respondents to be better absorbed into the skin. The results can be seen in FIGS. 1 and 2.
Overall the composition of formulation 1 was perceived to be a markedly preferred composition compared to formulation 4.
Three-quarters of the respondents rated the composition of formulation 1 as ‘very easy’ to apply (vs 16% for formulation 4)
Over four out of five rated formulation 1 as ‘absorbing well’ (vs 35% for formulation 4). 44% of respondents rated the skinfeel as ‘very pleasant’ (vs 11% for formulation 4).
The overall opinion of and quality of formulation 1 were also better. 39% rated formulation 1 as ‘very good’ overall (vs 8% for formulation 4)
44% rated the overall quality of formulation 1 as ‘very good’ (vs 5% for formulation 4.
The perceived effectiveness of formulation 1 was also well ahead of that for formulation 4.
56% rated it as ‘very effective’ (vs 11% for formulation 4).44% noted a ‘marked improvement’ after use (vs 10% for formulation 4).
The fact that Formulation 1 was preferred to formulation 4 in qualities such as absorption, overall quality and ease of application is very significant. It has been noted in the literature that the compliance of patients using creams is heavily linked to their perception of the composition. Where patients do not like the feel of the composition, they are reluctant to apply the composition in a sufficient quantity or often enough to achieve the available benefit. It is therefore very important to formulate a skin composition so as to appeal to the users.

What is claimed:

1. A semi-solid composition comprising emulsifier, oil and/or wax and from 10 to 15% by weight of sucrose palmitate, where the composition comprises a greater proportion of sucrose palmitate than of oil and/or wax and wherein the total amount of emulsifier in the composition is up to 20% of the composition for treating acne.

2. A composition according to claim 1 wherein the composition is a cream.

3. A composition according to claim 1 wherein the composition comprises more than one emulsifier.

4. A composition according to claim 1 wherein the composition comprises up to 15% of emulsifier.

5. A composition according to claim 1 wherein the ratio of sucrose palmitate to oil and/or wax is at most 2:1.

6. A kit for treating acne comprising a) a semi-solid composition comprising emulsifier, oil and/or wax and from 10 to 15% by weight of sucrose palmitate, where the composition comprises a greater proportion of sucrose palmitate than of oil and/or wax and wherein the total amount of emulsifier in the composition is up to 20% of the composition, together with instructions for simultaneous, concurrent, separate or sequential use in combination with b) a composition comprising a non-water-soluble sucrose fatty acid ester and a water-soluble sucrose fatty acid ester for treating acne.
